(19) United States
(12) Patent Application Publication
(10) Pub. No.: US 2011/0124600 A1
(43) Pub. Date: May 26, 2011

(54) Use of Dopamine partial agonists for treament of the restless leg syndrome and corresponding pharmaceutical preparation

(76) Inventors: Karel Kranda, Berlin (DE); Reinhard Horwski, Berlin (DE); Karel Sonka, Praha (CZ); Johannes Tack, Berlin (DE)

(21) Appl. No.: 10/517,435

(22) PCT Filed: Jun. 13, 2003
(86) PCT No.: PCT/EP03/06266

§ 371 Date: Jan. 24, 2011

(30) Foreign Application Priority Data
Jun. 13, 2002 (DE) 10226459.7

(51) Int. Cl. A61K 31/675; C07D 457/00; C07F 9/576; A61K 31/4375; A61P 21/00

(52) U.S. Cl. 514/89; 546/67; 546/22; 514/285

(57) ABSTRACT

Disclosed is the oral use of dopamine partial agonists and the physiologically acceptable salts thereof in order to create a pharmaceutical preparation for treating the restless legs syndrome, which effectively controls the symptoms while having a significantly smaller undesired effect of a medicament than medicaments known in prior art.

The invention relates to the use of dopamine partial agonists and their physiologically compatible salts and a corresponding pharmaceutical preparation for treatment of the restless leg syndrome.

The restless leg syndrome (RLS) is a neurological condition generally of the lower extremities with a prevalence of 9-15% of the adult population (H. Benes, offprint from “Der medizinische Sachverständige [The Medical Expert]”, 2000, Vol. 96, number 4, pp. 120-124). Since the symptoms tend to occur in the evening and it is often difficult for the patients to more accurately describe the symptoms, it can be assumed that a major portion of RLS patients have been incorrectly diagnosed to date. For roughly 10-15% of the patients who consult a physician due to sleep disturbances, RLS is diagnosed as the cause. It can be assumed that RLS is an often overlooked cause of insomnia or increased daytime fatigue.

RLS is a disease that has been recently described more often and that is characterized by serious sleep disturbances, motor hyperactivity, increased daytime fatigue, and a general reduction in the quality of life. The condition is characterized by unpleasant paresthesia in the legs that is described as “tingling, pulling, tearing, burning, or hurting” and that occurs almost exclusively in resting and relaxation situations. This paresthesia is associated with hyperkinesia of the legs that cannot be suppressed, with motor restlessness that forces the patient to continuously move or massage the legs or run around on them. Temporary relief or elimination of complaints by movement is characteristic. RLS occurs almost exclusively at night, which leads to a considerable adverse effect on the quality of sleep. This condition favors older patients, here up to 10% of the population (H. Benes, offprint from “Der medizinische Sachverständige,” 2000, Vol. 96, number 4, pp. 120-124). That RLS can be therapeutically influenced by dopaminergic substances is very reminiscent of Parkinson's disease (PD), a disease that is characterized by dopamine depletion of the substantia nigra. In fact, RLS can occur as a result of PD, but in most cases the cause is unclear (idiopathic RLS). In many cases, RLS can also occur as a result of an iron deficiency, a renal problem, hypothyroidism, a rheumatoid disease, vitamin B deficiency, a diabetic disease, or also a series of other diseases.

Seriously stricken patients often wander around all night and try to gain relief by physical measures such as showering, wrapping or massaging the legs. If they do then manage to fall asleep, the physiological course of sleep is generally seriously disrupted by so-called periodic leg movements during sleep (PMLS). They generally occur over long stretches of the night every 20-40 seconds.

Moreover, these RLS patients can only be employed to a limited degree for jobs with high demands for vigilance due to the often significant daytime fatigue with serious disruptions of the daytime mental state. The adverse effect on the quality of life, moreover, leads as far as to early retirement when there is no treatment or treatment is inadequate.

Just these properties of RLS and the problems resulting from the daytime fatigue caused by it illustrate the dimensions of the individual suffering and also the economic damage.

The causes and mechanisms of this disease for the most part have not yet been clarified. Various neuropharmacological studies support the assumption that central transmitter or receptor disturbances mainly of the dopaminergic/noradrenergic system play a part in etiopathological terms in the origin of RLS. At the focus are the endogenous opiate system and possibly other neurotransmitter systems that can be modulated by the dopaminergic system. Increased excitability of the monosynaptic and polysynaptic reflex arcs at the level of the brain stem and the spinal cord is assumed to be the trigger of RLS; this leads to reticularly controlled disinhibition phenomena in the descending reticulo-spinal system and is responsible both for paresthesias and also for motor phenomena. More recent functional MRT studies in RLS patients indicate the involvement of reticular structures near the midline in motor phenomena and an activity increase in the thalamus with sensory symptoms (Bucher et al., 1997, Ann. Neurol., 41, 639-645).

Successful treatment of RLS is critically necessary, especially since the dopaminergic therapies existing in the prior art are only partially effective, and, moreover, are subject to unpleasant side effects.

In a series of clinical studies, the therapeutic effectiveness of various active ingredients in RLS was studied. This group of substances includes levo-DOPA, generally in combination with a DOPA-decarboxylase inhibitor, and dopamine agonists from the group of ergot alkaloids, such as, e.g., bromocriptine and pergolide as well as dopamine agonists from the group of non-ergot alkaloids, such as, e.g., pramipexol dihydrochloride and ropinirole dihydrochloride. Moreover, opiates such as oxycodone and propoxyphene, benzodiapazines such as clonazepam, triazolam, nitrazepam and temazepam, anticonvulsive agents such as carbamazepine and gabapentin and other pharmaceutical agents of various classes, such as, e.g., propanolol, clonidine, baclofen, vitamins and minerals, have been tested.

The main problem in therapy with levo-DOPA was found to be the exacerbation of symptoms during the day and in the upper extremities. Levodopa exhibits brief effectiveness when it is administered perorally in the evening at a dosage from 100 to 600 mg. It is generally well tolerated, but, due to its short half-life, leads to suppression of symptoms only during the first part of the night. Chronic treatment with levodopa can lead to the aforementioned exacerbation of symptoms due to rebound phenomena.

Dopamine agonists from the group of ergot alkaloids are more effective than levo-DOPA, but their properties cannot be characterized so well in chronic use. An exact dosage is difficult due to the very great first-pass effect. Thus, e.g., only 6% of the perorally administered bromocriptine reaches systemic blood circulation. Moreover, interindividual and also intraindividual fluctuations with respect to the onset of action and also the duration of action are very dramatically pronounced. The very long plasma half-lives as well as the highly variable durations of action represent a major problem, therefore that of cabergoline is more than 40 hours, and that of pergolide is more than 27 hours. Sometimes, the administration of these dopaminergic pharmaceutical agents led to undesirable pharmaceutical agent effects such as confusion, anxiety states, restlessness, hallucinations, mental changes and double vision, which to some extent has led to termination of the medication.

Dopamine agonists from the group of non-ergot alkaloids likewise to some extent showed effects. Pramipexol that has a high affinity to D2 and D3 receptors in some patients led to almost complete suppression of symptoms, but treatment is often associated with gastrointestinal side effects such as nausea, constipation and loss of appetite, furthermore with dizziness and daytime fatigue. Ropinirole shows fewer side effects, but was also less effective.

Pharmaceutical agents from the group of opiates, such as, e.g., oxycodone and propoxyphene, and from the group of benzodiazepines, such as, e.g., clonazepam, triazolam, nitrazepam and temazepam, in part led to a remission of symptoms, but due to the known problems of this group of substances, such as tolerance or physical and psychological dependency, they can only be used to a very limited degree.

Anticonvulsive agents such as carbamazepine and gabapentin were especially effective in the control of painful RLS symptoms, while they showed only little effectiveness in the control of the remaining, mainly motor, RLS symptoms.

Pharmacotherapy of PD with dopaminergic pharmaceutical agents differs from RLS therapy in that in RLS therapy, the pharmaceutical substance is administered once a day, preferably in the evening. Conversely, in therapy of PD, a constant action level of these drugs is the goal. This difference explains why in RLS therapy, tolerance to typical dopaminergic side effects is generally observed less frequently. These greater fluctuations in blood plasma levels, however, also lead to side effects, such as, e.g., nausea, vomiting and orthostatic complaints occurring more frequently, possibly each evening. If in chronic RLS therapy, tolerance phenomena with respect to effectiveness should occur, the frequency of administrations must be increased, which in turn leads to more frequent occurrence of side effects.

In PD therapy, the use of dopamine and dopaminergic substances is reduced towards evening since it is known that the use of dopaminergic pharmaceutical substances can lead to disruption of sleep behavior and the sleep profile, and as a result, to a disturbance of REM phases, to daytime fatigue, daydreams and finally to hallucinations. Since in RLS therapy, dopaminergic substances are used exclusively in the evening, this can lead to enormous disruption of the sleep profile mainly at an overdosage. Thus, e.g., it was possible to show that the comparatively low dosage of 0.025 mg of lisuride intramuscularly, administered in the evening, led to significant disruption of the sleep profile in the first half of the night. It is obvious that these undesirable pharmaceutical agent effects in dopamine agonists with a longer half-life are even more strongly pronounced. As a result of the highly variable bioavailability, in some patients overdosages also repeatedly occur, while others are underdosed at the same dose. In summary, this means that the use of dopamine agonists in RLS therapy is very effective, but also engenders a whole series of problems such as the difficulty of finding the individual dose, with possible tolerance and overdosage. As a result, if bioavailability begins too quickly, symptoms such as nausea, vomiting and orthostatic problems often arise, as do sleep disturbances and daytime fatigue and cognitive problems, if there is an overdosage. It is obvious that the attempt to reduce undesirable effects by reducing the dosage administered generally leads to a reduction in the success of therapy.

An attempt to solve this problem was to administer dopamine agonists such as, e.g., lisuride transdermally, e.g., by using a patch. This form of administration, however, does not lead to complete suppression of undesirable pharmaceutical agent effects, and due to the longer latency phase has the major disadvantage that therapy is not effective in the cases in which symptoms begin in early evening and leads to the patients being unable to fall asleep.

The corresponding transdermal therapeutic systems are known from DE 100 43 321, DE 100 53 397 and DE 100 64 453, terguride also being able to be used.

The object of the invention is to make available a pharmaceutical agent for treatment of RLS that effectively controls the symptoms and has distinctly fewer undesirable pharmaceutical agent effects than the pharmaceutical agents that are known from the prior art.

This object is achieved by the features of claim 1.

For this purpose, according to the invention, the use of terguride (trans-dihydrolisuride, N,N-diethyl-N′-(8α)-6-methylergolin-8-yl)-urea), but also of cis-dihydrolisuride, cis-trans-dihydrolisuride and other terguride derivatives, such as, for example, 2-chloro-terguride, 2-chloro-lisuride or N1-allyl-terguride, and other dopamine partial agonists, such as SDZ 208-912 or preclamol ((−)3-PPP), is provided for producing a pharmaceutical agent for treatment of RLS, its being used solely orally. Furthermore, according to the invention, a pharmaceutical preparation is made available that is formulated for oral administration for treatment of RLS.

As physiologically compatible salts, salts of dopamine partial agonists with inorganic and organic acids are suitable. For example, hydrochloric acid, phosphoric acid, sulfuric acid, methane sulfonic acid, glucoheptanoic acid, succinic acid, tartaric acid, maleic acid, etc., are suitable for salt formation.

It has been found, surprisingly enough, that dopamine partial agonists and especially terguride are exceptionally effective in oral therapy of RLS and have a very prompt onset of action, but they do not have the typical side effects of the other dopaminergic substances, such as nausea, vomiting, and orthostatic disturbances, and even at a high dosage do not have any inherent adverse effects on the sleep profile, so that treatment of RLS that is almost free of side effects is possible by oral administration.

It was found that terguride at a dosage of 0.5 mg p.o., a concentration that is sufficient for control of RLS, up to a dosage of 50 mg, which corresponds to a clear overdosage, had no adverse effects whatsoever on the sleeping pattern, and primarily no adverse effect on REM phases. A dosage of 0.5-2.5 mg is preferred. In the therapy of RLS, terguride in higher dosages apparently acts as a partial agonist, its acting especially on RLS symptoms as a complete agonist, but has little or no effect on the other intact dopaminergic control circuits that control vomiting, orthostasis and the sleep profile. Thus, there is no need to specially change the dosage with a view to side effects. At the same time, however, the desired effect begins very quickly, which is very useful if RLS symptoms occur in the evening, and leads to the patient being unable to fall asleep. Finally, if a longer duration of action is desirable, the dosage of 0.5, 1 mg or even higher can be increased without disturbing the sleep profile.

It was further possible to show that terguride could also be used again in the cases in which it was stopped after RLS symptoms subsided, when the latter recurred. In this case, the terguride used showed the same effectiveness as in the first administration.

Finally, when symptoms occur in the course of the day, terguride can also be administered during the day in addition without loss of action and without the risk of the side effects of complete dopamine agonists.

In contrast to other dopaminergic substances, terguride has a positive effect on mood, cognitive performance and the daily activities of the patient, which can presumably be attributed to the clearly pronounced α2-adrenolytic properties of this substance. The positive effect of terguride on the general state of health of the patient produces very good compliance of the patient during therapy.

These α2-adrenolytic properties also lead to an improvement in the situation of patients suffering from benign prostatic hyperplasia. For the patients, this means a distinct improvement in sleep quality, since the symptoms of dysuria and polyuria are diminished.

The use according to the invention is also suited for long-term treatment since no physical and psychological dependency is to be expected.

All in all, terguride shows great effectiveness in the treatment of RLS, associated with very good tolerance.

The use of dopamine partial agonists according to the invention and especially of terguride and its derivatives takes place according to the invention solely as pharmaceutical agents for oral administration that can also have conventional vehicles and adjuvants. As additives and adjuvants, for example, binders, fillers, tabletting adjuvants, diluents, solubilizers, dyes, flavoring substances, wetting agents, emulsifiers, pH buffer additives, suspension adjuvants, non-aqueous adjuvants and preservatives can be used. Moreover, the dopamine partial agonist can also be combined with other pharmaceutical substances.

A filler can be selected from, for example, cellulose, mannitol and lactose. As solubilizers, for example, starch, starch derivatives and polyvinyl pyrrolidone can be used. The addition of EDTA to a solution of the active ingredient is advantageous. An emulsifier can be chosen from sodium lauryl sulfate, lecithin, sorbitan-monooleate and gum arabic. A suspension adjuvant can be chosen from, e.g., sorbitol, methyl cellulose, gelatin, hydroxyethyl cellulose, carboxymethyl cellulose, aluminum stearate gel, and hydrogenated edible fats. As non-aqueous adjuvants, i.a., almond oil, coconut oil, glycerol ester, propylene glycol, and ethyl alcohol are considered. A preservative can be chosen from methyl-p-hydroxybenzoate, ethyl-p-hydroxybenzoate, bisulfite and ascorbic acid. For example, magnesium stearate can be used as a lubricant.

The dosage of the pharmaceutical agent according to the invention is dependent on, i.a., the subject to be treated, the severity of the symptoms, and the type of administration. The effective dose for oral, sublingual, transdermal, rectal, topical and parenteral administration when using terguride is 0.5-50.0 mg/day, preferably, however, 0.5-2.5 mg/day.

Oral administration can be done, for example, in solid form, as tablets, capsules, granulations, powders, and lozenges, or in liquid form, as an aqueous solution, suspension, syrup, or soluble powder. Likewise, administration in the form of an oral spray is possible. The amount of active ingredient per oral administration unit when using terguride is 0.1-5.0 mg, preferably 0.5 mg.

Oral delayed-release forms that are obtained in the conventional manner, e.g. by adding hydrogenated fats and processing with resin-forming agents and coatings, as well as sublingual administration forms, are also suitable.

Drops for oral administration can be produced by aqueous solutions or suspensions of the active ingredient in oils with the addition of flavor-correcting agents, and/or solubilizers. At a daily dosage of 3×10 drops when using terguride, for example, 0.5-5 mg can be contained.

Other advantageous embodiments are characterized in the subclaims.

The invention is explained in more detail below based on examples in the form of patient histories.

### EXAMPLE 1

Patient 1, male, 62 years old, RLS diagnosed 20 years previously, no success with numerous attempts at treatment, at a daily dosage of 1.5 mg terguride orally shows outstanding objective and subjective effectiveness (RLS scores, 12 Epworth scale), now under long-term therapy, without loss of effectiveness or the necessity of increasing the dosage, no side effects.

### EXAMPLE 2

Patient 2, female, 45 years old, familial form of RLS for 30 years, previous treatment with biperiden of little effect, very distinct improvement with 1 mg of terguride orally, in a withdrawal trial immediate deterioration, during further therapy with 0.5 mg of partial effectiveness, no side effects; this patient was later switched to pergolide and showed no deterioration in doing so.

### EXAMPLE 3

Patient 3, 76 years old, male, RLS recognized for 9 years, magnesium, carbamazepine, zolpidem, and balneotherapy only moderately successful, has been orally treated for 3 months with 1 mg of terguride with very good success, no side effects.

1. Use of dopamine partial agonists and their physiologically compatible salts for producing a pharmaceutical agent for oral administration for treatment of the restless leg syndrome.

2. Use according to claim 1, characterized in that the dopamine partial agonists are selected from the following group:
terguride (trans-dihydrolisuride),
cis-dihydrolisuride,
dihydrolisuride (racemate),
2-chloro-terguride,
2-chloro-lisuride,
SDZ 208-912,
preclamol ((−)-3-PPP,
N1-allyl-terguride.

3. Use according to claim 2, wherein the terguride is administered in a dosage of 0.5-50 mg/day.

4. Use according to claim 3, wherein the terguride is administered in a dosage of 0.5-2.5 mg/day.

5. Use according to claim 1, wherein the dopamine partial agonist is used in combination with other pharmaceutical substances.

6. Pharmaceutical preparation for treatment of the restless leg syndrome, which contains at least one dopamine partial agonist or its physiologically compatible salts and which is formulated for oral administration.

7. Pharmaceutical preparation according to claim 6, wherein the dopamine partial agonists are selected from the following group:
terguride (trans-dihydrolisuride),
cis-dihydrolisuride,
dihydrolisuride (racemate),
2-chloro-terguride,
2-chloro-lisuride,
SDZ 208-912,
preclamol ((−)-3-PPP),
N1-allyl-terguride.

8. Pharmaceutical preparation according to claim 7, wherein the pharmaceutical preparation terguride contains a dosage of 0.5-50 mg/day.

9. Pharmaceutical preparation according to claim 8, wherein the terguride is administered in a dosage of 0.5-2.5 mg/day.

10. Pharmaceutical preparation according to claim 6, wherein the dopamine partial agonist is contained in combination with other pharmaceutical substances.

11. Pharmaceutical preparation according to claim 6, wherein the terguride is contained alone or in combination with galenical adjuvants.
